(19) United States
(12) Patent Application Publication
(10) Pub. No.: US 2004/0087553 A1
(43) Pub. Date: May 6, 2004

(54) Hypoestoxides, derivatives and agonists thereof for use in the treatment and prophylaxis of hyperlipidemia

(75) Inventors: Emeka J. Nchekwube, Morgan Hill, CA (US); Emmanuel A. Ojo-Amaize, Glendora, CA (US); Howard B. Cottam, Escondido, CA (US)

Correspondence Address: Pillsbury Winthrop LLP, Intellectual Property Group, P.O. Box 10500, McLean, VA, 22102, US

(73) Assignee: Paraquest, Inc., Bloomington, CA

(21) Appl. No.: 10/694,934

(22) Filed: Oct. 28, 2003

Related U.S. Application Data
(60) Provisional application No. 60/421,533, filed on Oct. 28, 2002.

(51) Int. Cl. A61K 31/665; A61K 31/335

(52) U.S. Cl. 514/100; 514/475

(57) ABSTRACT

Methods for treatment and prophylaxis of hyperlipidemias, including hypertriglyceridemia and hypercholesterolemia, are provided. The methods include administering to a host a therapeutically or prophylactically effective amount of a diterpene compound, such as a hypoestoxide.

This application claims the benefit of priority under 35 U.S.C. § 119 of provisional U.S. application Serial No. 60/421,533, filed Oct. 28, 2002, the contents of which are hereby incorporated by reference in their entirety, as if fully set forth.

### FIELD OF THE INVENTION

This invention relates to the use of diterpene compounds, in particular, hypoestoxides, derivatives and agonists thereof for treatment and prophylaxis of hyperlipidemias, including hypercholesterolemia and hypertriglyceridemia.

### BACKGROUND OF THE INVENTION

Hyperlipidemias are conditions of abnormal plasma lipids, lipoproteins, and/or cholesterol levels, and include hypercholesterolemia and hypertriglyceridemia. Hyperlipidemias commonly accelerate atherosclerosis and predispose individuals to coronary heart disease. Hyperlipidemias can be inherited conditions or can be the result of a lifestyle that includes dietary excess, increased body weight and little or no vigorous exercise. (Jay H. Stein et al., Eds., Internal Medicine, 5^{th} Ed., 1998, p. 1892.) References of interest providing background information on hyperlipidemia include: Foxton et al., Hyperlipidemia, Nursing Standard (Jun. 13, 1998) 12:49-56 and Krauss, Triglycerides and Atherogenic Lipoproteins: Rationale for Lipid Management, The American Journal of Medicine (Jul. 6, 1998) 105:58S-62S. All publications, patents, and patent documents referenced herein are incorporated by reference in their entirety as if fully set forth.

The plasma lipids cholesterol and triglycerides are insoluble in aqueous solutions and thus cannot circulate freely in plasma. Instead they are complexed with specialized proteins called apolipoproteins, or apoproteins. Lipid-apoprotein complexes are named lipoproteins and are produced by the gut and liver but are extensively modified in the plasma. The major function of lipoproteins is to transport lipids.

Hypertriglyceridemia (HTG) is a common inherited disorder of lipid metabolism in humans that is characterized by a proatherogenic lipoprotein profile, including increased plasma triglycerides and very low density lipoproteins (VLDL), and often decreased high density lipoproteins (HDL). Whereas its frequency in the general population is about 1% (1), HTG occurs in about 5% of patients surviving a myocardial infarction, indicating an increased risk for atherosclerosis. HTG can also be the result of dietary factors (excessive intake of total calories, carbohydrates and alcohol), diseases (e.g., diabetes mellitus and chronic renal failure) and drugs (e.g., oral contraceptives and beta-blockers). (Jay H. Stein et al., Eds., Internal Medicine, 5^{th} Ed., 1998, pp. 1894-1895.)

Moreover, a major component of atherosclerotic plaques is cholesterol; this cholesterol is believed to be derived largely from plasma cholesterol. Hypercholesterolemia is defined as a high plasma cholesterol level. The profile of individuals with hypercholesterolemia includes an elevated total cholesterol level and an elevated level of low-density lipoproteins (LDL). Individuals with severe hypercholesterolemia (total cholesterol over 300 mg/dl and LDL over 220 mg/dl) have a risk of coronary heart disease that is at least four times the baseline risk in the general population. (Jay H. Stein et al., Eds., Internal Medicine, 5th Ed., 1998, pp. 1892-1894.)

Further, hyperlipidemia is an important complication after organ transplantation and contributes to the development of post-transplant accelerated coronary artery diseases. Successful organ transplantation in humans requires the administration of pharmacologic immunosuppressants for prophylaxis of acute organ rejection. Cyclosporin A (CsA) and tacrolimus are now routinely used for transplantation of all solid organ and bone marrow transplantation. However, these agents are important causes of post-transplant hypertension, hyperlipidemia, and diabetes, all of which contribute to morbidity and mortality in the transplant recipient. (See, e.g., Akhlaghi, et al., Risk Factors for the Development and Progression of Dyslipidemia After Heart Transplantation, Transplantation (2002) 73:1258-1264.)

Because of their link with vascular disease, a number of approaches for controlling hyperlipidemias have been developed. Such approaches include changes in lifestyle, such as improved diet and increased exercise, as well as drug therapy. Drugs finding use in the management of plasma lipid profiles include: bile acid binding resins, niacin, HMG-CoA reductase inhibitors (statins) and fibric acid derivatives (e.g., gemfibrozil).

Despite the development of the above approaches, there continues to be a need for the identification of new treatment therapies for hyperlipidemias.

Examples of the use of diterpene compounds (e.g., hypoestoxides) for the treatment and prophylaxis of various conditions include U.S. Pat. No. 5,801,193 (inflammation, graft rejection, graft-versus-host disease and T-cell mediated autoimmune disorders), U.S. Pat. No. 6,242,484 (anti-parasitic therapy and prophylaxis), U.S. Pat. No. 5,994,328 (inhibiting tumor growth) and U.S. Pat. No. 6,001,871 (antiviral therapy), the contents of each of which are hereby incorporated by reference in their entirety, as if fully set forth.

### SUMMARY OF THE INVENTION

It is previously undisclosed in the art to use hypoestoxides and other diterpene compounds for the treatment and prophylaxis of hyperlipidemias. Thus, the present invention provides methods of treating a host, such as a human, suffering from hyperlipidemia resulting from elevated plasma levels of cholesterol, triglycerides and/or lipoproteins such as VLDL, with hypoestoxides, derivatives and agonists thereof, such that those pathological condition(s) are ameliorated thereby. In addition, methods of prophylaxis against the development of hyperlipidemias commonly observed in recipients of solid organ or bone marrow transplants are provided. Thus, the method comprises administering to the afflicted host or transplant recipient a therapeutically, or prophylactically, effective amount of a compound having the formula I:

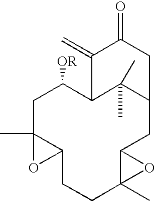

or pharmaceutically acceptable salts thereof, wherein R is:

a) H or acetyl,

b) P(O)(OH)₂,

c) P(O)(OH)(OM), wherein M is selected from the group consisting of an alkali metal salt and an alkaline earth metal salt,

d) P(O)OM₂ wherein M is each independently selected from the group consisting of alkali metal salts and alkaline earth metal salts,

e) Alkyl of 1 to 12 carbon atoms having 0 to 6 double bonds, said alkyl selected from the group consisting of substituted, unsubstituted, straight chain and branched alkyls,

f) (CH₂)n morpholine, wherein n=1-4,

g) morpholinomethylphenyl, ortho-aminophenyl or ortho-hydroxyphenyl,

h) (CH₂)n COOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, or

i) COR₁ wherein R₁ is selected from the group consisting of H, (CH₂)n CH₃ wherein n=0-6, (CH₂)n COOR₂ wherein n=1-4 and R₂ is each selected from the group consisting of H, an alkali metal salt, an alkaline earth metal salt, NH₄+ and N+(R₃)₄, and (CH₂)n N+(R₃)₄, wherein n=1-4 and R₃ is each independently selected from the group consisting of H and an alkyl of 1 to 4 carbon atoms,

and wherein the effective amount is an amount sufficient to ameliorate at least one symptom of said disease, and compounds may be used alone or in combination with other chemotherapeutic agents.

### DETAILED DESCRIPTION OF THE INVENTION

Methods of treating a host suffering from hyperlipidemia are provided. In the subject methods, an effective amount of an agent as described above is administered to an afflicted host, in an amount sufficient to ameliorate at least one condition related to or included under the definition of hyperlipidemias. As used herein, the term “host” or “subject” is taken to mean human, as well as other animals. The term “ameliorate” means to improve, lessen the severity of or mitigate. Also provided are methods for prophylactically treating a host patient at risk of a hyperlipidemic condition, such as a transplant patient, with an agent as described above, in combination with standard chemotherapeutic agents, such as CyA or tacrolimus. The methods comprise administering to the afflicted host or host patient a therapeutically, or prophylactically, effective amount of a compound having the formula I:

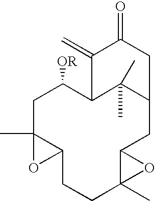

or pharmaceutically acceptable salts thereof, wherein R is:

a) H or acetyl,

b) P(O)(OH)₂,

c) P(O)(OH)(OM), wherein M is selected from the group consisting of an alkali metal salt and an alkaline earth metal salt,

d) P(O)OM₂ wherein M is each independently selected from the group consisting of alkali metal salts and alkaline earth metal salts,

e) Alkyl of 1 to 12 carbon atoms having 0 to 6 double bonds, said alkyl selected from the group consisting of substituted, unsubstituted, straight chain and branched alkyls,

f) (CH₂)n morpholine, wherein n=1-4,

g) morpholinomethylphenyl, ortho-aminophenyl or ortho-hydroxyphenyl,

h) (CH₂)n COOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, or

i) COR₁ wherein R₁ is selected from the group consisting of H, (CH₂)n CH₃ wherein n=0-6, (CH₂)n COOR₂ wherein n=1-4 and R₂ is each selected from the group consisting of H, an alkali metal salt, an alkaline earth metal salt, NH₄+ and N+(R₃)₄, and (CH₂)n N+(R₃)₄, wherein n=1-4 and R₃ is each independently selected from the group consisting of H and an alkyl of 1 to 4 carbon atoms,

and wherein the effective amount is an amount sufficient to ameliorate at least one symptom of said disease, and compounds may be used alone or in combination with other chemotherapeutic agents.

Preferred embodiments of the invention are compounds of formula I, wherein R═H or R=acetyl (hypoestoxide).

The magnitude of a prophylactic or therapeutic dose of compounds of formula I in the treatment or prevention of hyperlipidemia may vary with the progression of the disease, the chemotherapeutic agent(s) or other therapy used, and the route of administration. The dose, and perhaps the dose frequency, may also vary according to the age, body weight, and response of the individual patient. In general, the total daily dose range for compounds of formula I, for the conditions described herein, may be from about 0.5 mg to about 5000 mg, in single or divided doses. Preferably, a daily dose range may be about 1 mg to about 4000 mg, in single or divided doses. In managing the patient, the therapy may be initiated at a lower dose and may be subsequently increased depending on the patient's global response. Patients, including but not limited to, infants, children, patients over 65 years, and those with impaired renal or hepatic function may initially receive lower doses. Doses for these patients may be titrated based on global response and blood level. It is possible to use dosages outside these ranges in some cases. Further, it is noted that it will be readily apparent to the clinician or treating physician how and when to interrupt, adjust or terminate therapy in conjunction with individual patient response. The term “an effective amount” is meant to encompass the above-described dosage amounts and dose frequency schedule.

Any suitable route of administration may be employed for providing the patient with an effective amount of compounds of formula I. For example, and without limitation, oral, rectal, parenteral (subcutaneous, intravenous, intramuscular), intrathecal, transdermal, and similar forms of administration may be employed. Dosage forms may include tablets, troches, dispersions, suspensions, solutions, capsules and patches. The compound may be administered prior to, concurrently with, or after administration of other chemotherapy, or continuously (i.e., in daily doses, during all or part of, a chemotherapy regimen, such as a CsA regimen.) The compound, in some cases, may be combined with the same carrier or vehicle used to deliver the other chemotherapeutic agent.

Thus, the compounds of the present invention may be systemically administered (e.g., orally, in combination with a pharmaceutically acceptable vehicle such as an inert diluent or an assimilable edible carrier). The compounds of the present invention may be enclosed in hard or soft shell gelatin capsules, may be compressed into tablets, or may be incorporated directly with the food of the patient's diet. For oral therapeutic administration, the active compound may be combined with one or more excipients and used in the form of, inter alia, ingestible tablets, buccal tablets, troches, capsules, elixirs, suspensions, syrups or wafers. Such compositions and preparations may contain at least 0.1% of an active compound of the present invention. The percentage of the compositions and preparations may, of course, be varied and may conveniently be between about 2 to about 60% of the weight of a given unit dosage form. The amount of active compound in such therapeutically or prophylactically useful compositions is such that an effective dosage level will be obtained.

The tablets, troches, pills, capsules, and the like may also contain the following: binders such as gum tragacanth, acacia, corn starch or gelatin, excipients such as dicalcium phosphate, disintegrating agents such as corn starch, potato starch and alginic acid, lubricants such as magnesium stearate, sweetening agents such as sucrose, fructose, lactose or aspartame, or a flavoring agent such as peppermint, oil of wintergreen, or cherry flavoring. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with, inter alia, gelatin, wax, shellac or sugar. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a non-toxic surfactant. Dispersions can also be prepared in glycerol, liquid polyethylene glycols, triacetin, and mixtures thereof and in oils. Under ordinary conditions of storage and use, these preparations may contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, non-toxic glyceryl esters, and suitable mixtures thereof. The proper fluidity can be maintained, such as, for example, by the formation of liposomes, by the maintenance of the required particle size in the case of dispersions or by the use of surfactants. The prevention of the action of microorganisms can be brought about by various antibacterial and antifungal agents (e.g., parabens, chlorobutanol, phenol, sorbic acid and thimerosal). In many cases, it may be preferable to include isotonic agents, for example, sugars, buffers or sodium chloride. Prolonged absorption of the injectable compositions can be brought about by the use in the compositions of agents delaying absorption, such as, for example, aluminum monostearate and gelatin.

Sterile injectable solutions of the compounds of the present invention may be prepared by incorporating the active compound in the required amount in an appropriate solvent with various other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

Useful dosages of the compounds of formula I may be determined by comparing their in vivo activity in animal models. Methods for the extrapolation of effective dosages in mice and other animals to humans are known in the art (see, e.g., U.S. Pat. No. 4,938,949).

### EXAMPLES

In one example, hypoestoxide was administered orally to a 2-year-old female beagle dog at a dose of 30 mg/kg, once daily for seven days. Blood cholesterol and triglyceride levels were determined in sera obtained at days 1, 3, 7 and 14, respectively. Assays were performed by a standard colorimetric method by a commercial laboratory (Antech Diagnostics, Irvine, Calif.). The administration of hypoestoxide significantly lowered blood cholesterol and triglyceride levels, as shown in Table 1.

TABLE 1
The effect of oral administration of hypoestoxide on blood
cholesterol and triglyceride levels in dogs

| | Days | | | | | Normal |
|---|---|---|---|---|---|---|
| | −1 | 1 | 3 | 7 | 14 | Reference Range |
| Cholesterol | 308 | 122 | 130 | 124 | 134 | (92-324) |
| (mg/dl) | | | | | | |
| Triglycerides | 300 | 296 | 70 | 33 | 51 | (29-291) |
| (mg/dl) | | | | | | |

In another example, oral ingestion of a 1 g capsule of dried leaf powder of hypoestes rosea shrub (parent plant of hypoestoxide), taken once daily for one year as a dietary supplement, significantly lowered blood cholesterol and triglyceride levels in a human subject, as shown in Table 2.

TABLE 2
The effect of oral ingestion by humans of dried leaf
powder of hypoestes rosea shrub, containing 0.1% hypoestoxide

| Year→ | −1 | 1 | Normal Reference Range |
|---|---|---|---|
| Cholesterol (mg/dl) | 215 | 206 | (50-200) |
| Triglycerides (mg/dl) | 103 | 96 | (30-210) |

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method of treating a host having hyperlipidemia comprising administering to the host an effective amount of a compound having the formula: 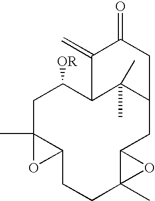
and pharmaceutically acceptable salts thereof, wherein R is selected from the group consisting of:
a) H or acetyl,
b) P(O)(OH)₂,
c) P(O)(OH)(OM), wherein M is selected from the group consisting of an alkali metal salt and an alkaline earth metal salt,
d) P(O)OM₂ wherein M is each independently selected from the group consisting of alkali metal salts and alkaline earth metal salts,
e) Alkyl of 1 to 12 carbon atoms having 0 to 6 double bonds, said alkyl selected from the group consisting of substituted, unsubstituted, straight chain and branched alkyls,
f) (CH₂)n morpholine, wherein n=1-4,
g) morpholinomethylphenyl, ortho-aminophenyl or ortho-hydroxyphenyl,
h) (CH₂)n COOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, and
i) COR₁ wherein R₁ is selected from the group consisting of H, (CH₂)n CH₃ wherein n=0-6, (CH₂)n COOR₂ wherein n=1-4 and R₂ is each selected from the group consisting of H, an alkali metal salt, an alkaline earth metal salt, NH₄+ and N+(R₃)₄, and (CH₂)n N+(R₃)₄, wherein n=1-4 and R₃ is each independently selected from the group consisting of H and an alkyl of 1 to 4 carbon atoms.

2. The method of claim 1 wherein the compound is used in combination with other chemotherapeutic agents.

3. The method of claim 1 wherein R is selected from the group consisting of H and acetyl.

4. The method of claim 3 wherein the hyperlipidemia is selected from the group consisting of hypertriglyceridemia and hypercholesterolemia.

5. The method of claim 1 wherein the daily dose range of the compound is from about 0.5 mg to about 5000 mg.

6. The method of claim 1 further including incorporating the compound in a dosage form selected from the group consisting of a tablet, a troche, a dispersion, a suspension, a solution, a capsule, a patch, a syrup, an elixir and a wafer.

7. The method of claim 6 wherein the dosage form contains at least 0.1% by weight of the compound.

8. A method for protecting a host from developing hyperlipidemia comprising administering to the host an effective amount of a compound having the formula: 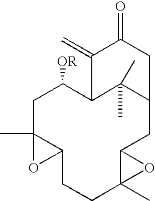
and pharmaceutically acceptable salts thereof, wherein R is selected from the group consisting of:
a) H or acetyl,
b) P(O)(OH)₂,
c) P(O)(OH)(OM), wherein M is selected from the group consisting of an alkali metal salt and an alkaline earth metal salt,
d) P(O)OM₂ wherein M is each independently selected from the group consisting of alkali metal salts and alkaline earth metal salts,
e) Alkyl of 1 to 12 carbon atoms having 0 to 6 double bonds, said alkyl selected from the group consisting of substituted, unsubstituted, straight chain and branched alkyls,
f) (CH₂)n morpholine, wherein n=1-4,
g) morpholinomethylphenyl, ortho-aminophenyl or ortho-hydroxyphenyl,
h) (CH₂)n COOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, and
i) COR₁ wherein R₁ is selected from the group consisting of H, (CH₂)n CH₃ wherein n=0-6, (CH₂)n COOR₂ wherein n=1-4 and R₂ is each selected from the group consisting of H, an alkali metal salt, an alkaline earth metal salt, NH₄+ and N+(R₃)₄, and (CH₂)n N+(R₃)₄, wherein n=1-4 and R₃ is each independently selected from the group consisting of H and an alkyl of 1 to 4 carbon atoms.

9. The method of claim 8 wherein the compound is used in combination with other chemotherapeutic agents.

10. The method of claim 9 wherein the other chemotherapeutic agents are selected from the group consisting of Cyclosporin A and tacrolimus.

11. The method of claim 8 wherein R is selected from the group consisting of H and acetyl.

12. The method of claim 8 wherein said host is at risk for developing hyperlipidemia due to recent solid organ or bone marrow transplantation.

13. The method of claim 8 wherein the daily dose range of the compound is from about 0.5 mg to about 5000 mg.

14. The method of claim 8 further including incorporating the compound in a dosage form selected from the group consisting of a tablet, a troche, a dispersion, a suspension, a solution, a capsule, a patch, a syrup, an elixir and a wafer.

15. The method of claim 14 wherein the dosage form contains at least 0.1% by weight of the compound.
